(19) United States
(12) Patent Application Publication
(10) Pub. No.: US 2005/0148879 A1
(43) Pub. Date: Jul. 7, 2005

(54) Mode dependent tunable transducers and methods of use

(75) Inventors: Bhaskar Ramamurthy, Los Altos, CA (US); Sevig Ayter, Cupertino, CA (US); Kutay F. Ustuner, Mountain View, CA (US); Henry G. Pavy, Cupertino, CA (US); John D. Marshall, Campbell, CA (US)

Correspondence Address: Siemens Corporation;Intellectual Property Department, 170 Wood Avenue South, Iselin, NJ, 08830, US

(73) Assignee: Siemens Medical Solutions USA, Inc.

(21) Appl. No.: 10/741,958

(22) Filed: Dec. 19, 2003

(51) Int. Cl. A61B 8/14

(52) U.S. Cl. 600/459

(57) ABSTRACT

Transducer systems for mode dependent tuning and associated methods are provided. One or more tuning circuits are provided within a transducer probe. The tuning circuit is switchably connected to the transducer. By connecting or disconnecting the tuning circuit, the tuning for the transducer element is varied. Selective tuning of a medical ultrasound transducer allows different tuning for different modes of operation. For example, the frequency response of the transducer is varied between different modes of imaging, such as B-mode and flow-mode imaging. Higher frequency signals are used for higher resolution B-mode imaging, but a stronger response at lower frequency is desired for better penetration during flow-mode imaging and Doppler modes. Mode is used in a general sense, such as associated with an imaging mode as well as a type of operation (e.g. transmit mode versus receive mode operation).

### BACKGROUND

The present invention relates to transducers. In particular, tuning an ultrasound or other transducer to imaging system signals is provided.

Medical diagnostic ultrasound transducers are tuned to the system to provide sensitivity over a wide bandwidth. Typically, the tuning is fixed. Inductors, capacitors or resistances provided by various components of the imaging system and transducer as well as added components are used to tune the transducer elements to the beamformer channels. The added elements, such as an inductor, have fixed values.

The tuning may be variable. U.S. Pat. No. 3,980,905 discloses a fixed tuning network connected with an amplifier having a variable output impedance. By varying the output impedance of the amplifier, the response of the transducer is tuned or varied. U.S. Pat. No. 6,416,478 discloses a multi-layer transducer element. A low pass, band pass or high pass filter is switched on for one of transmit or receive operation. The filter is isolated or switched off for other operation. The filtering circuit, such as a circuit including a capacitance, affects the tuning of the transducer elements. Other variable tuning circuits provide fixed tuning for transmit operation and a variable tuning within the ultrasound system for receive operation. The tuning is switchable between beams, so may vary as a function of imaging mode. In another device, fixed receive only tuning is provided in a transducer probe housing using z1 and z2 elements of the structure described in U.S. Pat. No. 6,269,052, the disclosure of which is incorporated herein by reference.

### BRIEF SUMMARY

By way of introduction, the preferred embodiments described below include transducer systems for mode dependent tuning and associated methods. One or more tuning circuits are provided within a transducer probe. The tuning circuit is switchably connected to the transducer. By connecting or disconnecting the tuning circuit, the tuning for the transducer element is varied. Connections may also be provided to choose different tuning circuits. Selective tuning of a medical ultrasound transducer allows different tuning for different modes of operation. For example, the frequency response of the transducer is varied between different modes of imaging, such as B-mode and flow-mode imaging. Higher frequency signals are used for higher resolution B-mode imaging, but a stronger response at lower frequency is desired for better penetration during flow-mode imaging. Mode is used in a general sense, such as associated with an imaging mode as well as a type of operation (e.g. transmit mode versus receive mode operation). Although the discussion below refers primarily to tuning networks, other networks including but not limited to preamplifiers, delay lines and pulse shapers may be switched into and out of the transducer path using the means described herein.

In a first aspect, a transducer system for mode dependent tuning is provided. A transducer element has a first element. First and second tuning circuits are electrically connectable with the first element. Each of the tuning circuits has at least a capacitor, resistor, inductor or combinations thereof. A switch is operable to connect and disconnect at least one of the first and second tuning circuits to the first element.

In a second aspect, a transducer system for mode dependent tuning is provided. First and second tuning circuits are electrically connectable with a transducer element. A switch is operable to connect and disconnect at least one of the tuning circuits to the transducer element. A controller is operable to control the switch to connect the one tuning circuit to the transducer element during both transmit and receive operations of a first mode and to disconnect the one tuning circuit from the transducer element during both transmit and receive operations of a second, different mode.

In a third aspect, a transducer system for mode dependent tuning is provided. First and second tuning circuits are electrically connectable with a single layer transducer element. A switch is operable to connect and disconnect at least one of the tuning circuits to the single layer transducer element.

In a fourth aspect, a transducer system for mode dependent tuning is provided. First and second tuning circuits are electrically connectable with a transducer element. A switch is operable to connect and disconnect at least one of the tuning circuits to the transducer element. A controller is operable to control the switch to connect the one of the tuning circuits to the transducer element during transmit operation of a first mode and to disconnect the one of the tuning circuits from the transducer element during a transmit operation of a second, different mode.

In a fifth aspect, a method for mode dependent tuning of a transducer is provided. During operation in a first mode, a first circuit in a transducer is connected to a transducer element. During operation in a second mode, the first circuit is disconnected from the transducer element. During the operation in the two different modes, one of: (1) transmitting in the first mode and transmitting in the second mode or (2) transmitting in the first mode and receiving the second mode with the transducer element being a single layer element is provided.

In a sixth aspect, a method for mode dependent tuning of a transducer is provided. A single layer transducer element is tuned with a first tuning circuit during a first mode. The first tuning circuit is connected between a cable and the single layer transducer element. A single layer transducer element is tuned with a second tuning circuit during a second mode different than the first mode. The second tuning circuit is different than the first tuning circuit. The second tuning circuit is connected between the cable and the single layer transducer element.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a transducer system for mode dependent tuning;

FIG. 2 is a circuit diagram of another embodiment of a transducer system for mode dependent tuning;

FIG. 3 is a graphical representation of the spectral content of various possible tunings of a transducer; and

FIG. 4 is a flowchart diagram of one embodiment of a method for mode dependent tuning of a transducer.

### DETAILED DESCRIPTION OF THE DRAWINGS AND PRESENTLY PREFERRED EMBODIMENTS

Switching circuitry for tuning a transducer as a function of mode of operation is integrated within the transducer probe housing or adjacent (e.g., on the same substrate as or attached to the probe of the transducer) to the transducer elements. The switching circuitry and associated connectable tuning circuitry are connected between a cable and a transducer element. Alternatively, the circuitry is connected within a part of the transducer assembly, such as in a housing for a connector for connection to an ultrasound system or in the transducer probe housing. By selecting one of multiple possible tuning circuits, the frequency response of a transducer element is varied. Varied frequency response is used for different modes of operation of the system.

FIG. 1 shows one embodiment of a transducer system 10 for mode dependent tuning. The transducer system 10 includes a transducer element 12, two tuning circuits 14 and 16, one or more switches 18, a cable 20 and a controller 22. Different, fewer or additional components may be provided. For example, additional elements 12 in a transducer array are provided for connection with the same or different cables 20. As another example, additional tuning circuits 14, 16 and switches 18 are provided. The system 10 is provided for each element 12 of a transducer array or used for only a subset of the elements of a transducer array. The controller 22 is common to all of the systems 10 and associated elements or separate controllers 22 are provided for subsets or individual element systems. In one embodiment, the system 10 is used for a medical diagnostic ultrasound imaging system transducer. In other embodiments, the system 10 is part of a mechanical testing ultrasound transducer or any other sonic transducer.

The switches 18, tuning circuits 14 and 16 and transducer element 12 are housed within a probe housing in one embodiment. The probe housing encloses the transducer elements, such as a handheld scanhead. In other embodiments, the tuning circuits 14 and/or 16 and/or switches 18 are within the transducer connector that connects to the system. Additional tuning circuits and associated transducer elements may be housed within the same probe housing or at the imaging system. For example, a one dimensional, two dimensional or other array of elements is provided within the probe housing. The cable 20 connects each of the elements 12 from the probe housing to an ultrasound imaging system. The controller 22 is within the ultrasound imaging system, but may be positioned within the probe housing or connector of the transducer assembly.

The transducer element 12 is a piezoelectric, microelectromechanical, capacitive membrane, or other now known or later developed material or device for transducing between electrical and acoustical energies. The transducer element 12 has a plurality of electrodes 24. One of the electrodes 24 connects with the switch 18 or one or more of the tuning circuits 14, 16. The connection is either direct or indirect through one or more additional components. The other electrode 24 connects to a virtual ground. In alternative embodiments, different connections are provided for the electrodes 24. In one embodiment, the transducer element 12 is a single layer element, having a single layer of transducer material. In alternative embodiments, the transducer element 12 has multiple layers of transducer material separated by one or more electrodes. The layers are arranged along a depth or range dimension so that acoustic energy generated by a lower of the two elements is transmitted through an upper layer and acoustic energy incident upon the element from a patient passes through the upper layer to the lower layer. A delay or other tuning may be switched for one layer and not the other layer or for both layers together. Any of various now known or later developed transducer element configurations or stacks may be used.

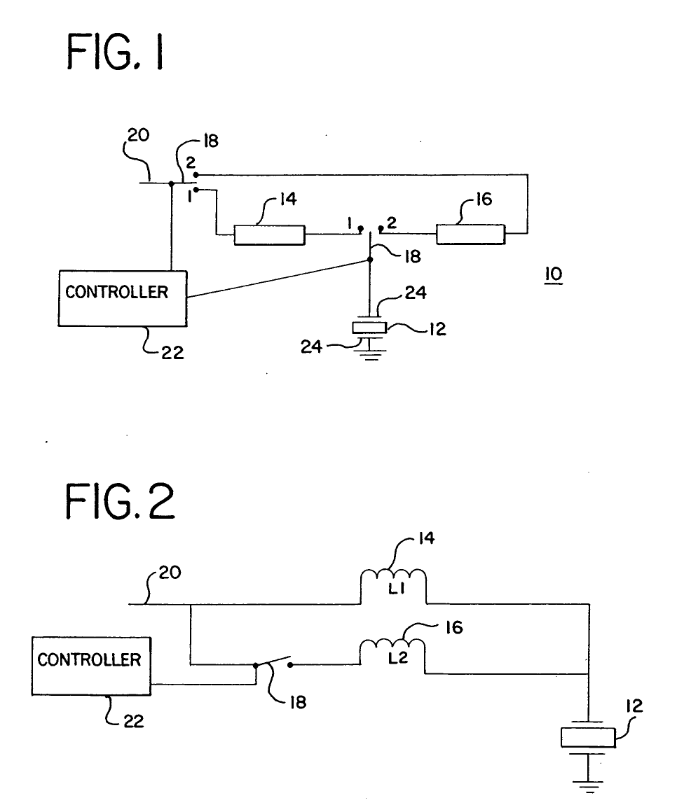

The tuning circuits 14 and 16 have at least one of a capacitor, a resistor, an inductor and combinations thereof. Additional components may be provided for each of the tuning circuits 14, 16. In one embodiment, each of the tuning circuits 14 and 16 have a same type of component, such as a single inductor as shown in FIG. 2. The same inductor or type of component has a different inductance or value. For example, the inductor L1 of FIG. 2 has an 18 μH and the parallel inductance L2 of the second tuning circuit 16 has a 10 μH inductance. In other embodiments, one of the tuning circuits 14 has a different type, different combination, different number, different values and combinations thereof of components than the other tuning circuit 16. In yet another alternative embodiment, the tuning circuits 14, 16 share at least one common component, such as a resistance provided by the system, a resistor, a capacitor, an inductor or other component and have at least one different component. In yet another alternative embodiment, one of the tuning circuits 14, 16 is a signal trace or a line without further analog or digital components.

The electrode 24 or the transducer element 12 electrically connects with the tuning circuits. Electrically connectable is provided by a permanent electrical connection to the transducer element 12 or by a switchable connection. For example, the first tuning circuit 14 of FIG. 2 is connected to the transducer element 12 without switches and the second parallel inductor 16 is also connected to the transducer element 12 but switchably connected in parallel with the cable 20. As yet another example, each of the tuning circuits 14 and 16 of FIG. 1 are switchably connected and disconnected from the transducer element 12 and the cable 20. Other switching arrangements may be provided.

The switch or switches 18 are one or more of relays, multiplexer, transistors, analog switches, digital switches, diode switches, microelectromechanical switches or other now known or later developed devices for selecting between two or more electrical signal lines or components. Where multiple switches 18 are provided, each of the switches 18 is a same or different type of switch. In one embodiment, the switch 18 is a relay or microelectromechanical switch implemented on a semiconductor substrate with a capacitive membrane ultrasound transducer element. Alternatively, the switch 18 is implemented on a different substrate than the transducer element 12. In another embodiment, the switch 18 is a pair of opposite polarity diodes or Zener diodes. Greater voltages switch the diodes on allowing a greater voltage signal to pass while the diodes block lesser voltages. For example, the switch is automatically turned on by greater transmit voltages but remains off for lesser receive operation voltages. The switch 18 is positioned in series with the tuning circuits 14 and 16, such as shown for both switches 18 of FIG. 1. Alternatively, the switch 18 is positioned in parallel with one of the tuning circuits and in series with the other tuning circuit 16 as shown in FIG. 2. In yet other alternative embodiments, one switch is configured in parallel with one of the tuning circuits 14, 16 and another switch is configured in series with both tuning circuits 14, 16. Any other possible configuration of parallel and/or series connections of one or more switches 18 and one or more tuning circuits 14 and 16 is possible. Additional switches may be provided for use with additional tuning circuits.

Similarly, it is possible to fabricate a circuit element that presents a very small impedance to low level signals but strongly limits current flow from large signals. See for example U.S. Pat. No. 6,269,052, the disclosure of which is incorporated herein by reference. Using the circuit elements described above for voltage limiting (the double diode element) and for current limiting, it is possible to design active components (e.g., transistors, amplifiers, or processors) that switch automatically between transmit and receive modes and/or provide different functions for the same or different imaging modes. The available functions include receive-only or transmit-only tuning networks, receive-only preamplifiers or any other circuits to modify the signal path characteristics in one mode but not modify or modify differently the signal path in the other mode.

In the configuration shown in FIG. 1, the switches 18 are operable to disconnect one tuning circuit 16 from the transducer element 12 and the cable 20 when the other tuning circuit 14 is connected to the transducer element 12 and the cable 20. The switches 18 are also operable to disconnect the tuning circuit 14 from the transducer element 12 and the cable 20 when the second tuning circuit 16 is connected to the transducer element 12 and the cable 20. Alternatively, the switches 18 select one of the tuning circuits 14, 16 for connection through various modes of operation to the transducer element 12. The other tuning circuit 16 is disconnected or connected to the transducer element 12 and the tuning circuit 14 as a function of mode of operation. In this case, separate switches 18 are used for each of the tuning circuits 14, 16. Alternatively, the circuit arrangement shown in FIG. 2 is used for keeping one tuning circuit 14 connected to the transducer element while selectively connecting the other tuning circuit 16. In either configuration, at least one of the tuning circuits 14, 16 is connected or disconnected from the transducer element 12 or cable 20 as a function of the mode of operation.

The cable 20 is a coaxial cable, twisted pair, conductor, or other now known or later developed device for transmitting electrical signals between the medical imaging system and the transducer element 12. In one embodiment, a miniaturized coaxial cable 20 is provided for each transducer element, but a coaxial cable may be shared between multiple elements, such as using a time division or other multiplexing. The cable 20 connects with one or more switches 18. The switch 18 and at least part of the tuning circuits 14, 16 are connectable between the cable 20 and the transducer element 12 and associated electrode 24. The cable 20 is electrically and physically connectable with an imaging system and extends from the imaging system to the transducer housing or probe.

The controller 22 is a processor, digital signal processor, application-specific integrated circuit, memory, analog circuit, digital circuit, FPGA, combinations thereof or other now known or later developed device for controlling one or more switches 18. In one embodiment, the controller 22 is part of the beamformer controller structure of an ultrasound imaging system. In another embodiment, the controller 22 is part of an overall system controller, but the controller 22 may be distributed or centralized in any of various locations within the imaging system. The controller 22 is connectable with the switches 18, such as by multiplexing a control signal on the cable 20. Alternatively, a separate control path is provided from the imaging system to the controller 22 or switches 18. For example, the controller 22 is positioned within the imaging system and an extra cable or control line is provided for controlling the switches 18.

The controller 22 is operable to control the switches 18 as a function of a mode of operation of the transducer element 12. The mode is used broadly to include transmit versus receive operation. The mode also includes a type of imaging mode, such as operation for B-mode, flow mode (e.g., Doppler mode), harmonic mode, M-mode, spectral Doppler mode, three dimensional imaging mode, combinations thereof or other now known or later developed imaging modes. For example, the controller 22 is operable to switch in one tuning circuit 14 by controlling the switches 18 for B-mode imaging at higher frequencies, and operable to switch to an additional or different tuning circuit 16 for operation at lower frequencies for flow mode. Lower frequencies may allow greater penetration for flow mode or Doppler imaging, and higher frequencies may provide for higher resolution imaging for B-mode imaging. Frequently, multiple modes are used for scanning a patient at a same or substantially same time. For example, during real time imaging, a patient is sequentially scanned in two different modes. For example, B-mode acquisitions are performed along one or more scan lines followed by flow mode, harmonic mode, M-mode or spectral Doppler mode transmissions along one or more scan lines. The two modes and tuning are interleaved sequentially during the imaging session.

In one embodiment, the controller 22 is operable to control one or more switches 18 such that a particular one or combination of the tuning circuits 14 and 16 are connected with the electrode 22 and associated transducer element 12 during receive operation and a different one or combination is connected during transmit operation. For example, one of the tuning circuits 14, 16 is connected to the single layer transducer element 12 during a transmit or receive operation and disconnected during the other of the transmit and receive operation. The other tuning circuit 16 is either connected during both transmit and receive operation or connected and disconnected at a same time as the tuning circuit 14, or the tuning circuit 16 is disconnected while the tuning circuit 14 is connected and is connected while the tuning circuit 14 is disconnected. Any of various combinations of connections and disconnections of tuning circuits 14, 16 during different transmit or receive mode operation may be used.

In another embodiment, the controller 22 is operable to control one or more switches 18 such that one tuning circuit 14 connects with the electrode 24 for transmit operation in a first imaging mode and such that the other tuning circuit 16 connects with the electrode 24 for a transmit operation in a second imaging mode different than the first imaging mode. When the tuning circuit 14 is connected, the tuning circuit 16 is either also connected or disconnected. Likewise, when the tuning circuit 16 is connected, the tuning circuit 14 is either connected or disconnected. One or more tuning circuits, such as the tuning circuit 16, are connected during both imaging modes. For example, FIG. 2 shows a circuit where the tuning circuit 16 is connectable and disconnectable as a function of the imaging mode or other mode of operation while the tuning circuit 14 is always connected regardless of the mode of operation. Alternatively, FIG. 1 shows tuning circuits 14, 16 that may be selectively connected and disconnected as a function of imaging mode or for other reasons.

In another embodiment, the controller 22 is operable to control the switches 18 such that a same tuning circuit or combination of tuning circuits 14, 16 is connected with the electrode 24 and associated element 12 during both transmit and receive operations of a first mode of operation, and a different one of and/or combination of tuning circuits 14, 16 is connected during both transmit and receive operations of a second mode of operation different than the first mode. For example, one of the tuning circuits 16 is connected during transmit and receive operation for a first imaging mode and disconnected during transmit and receive operation for the second imaging mode. The other tuning circuit 14 is either connected at a same time, disconnected when the other tuning circuit 16 is connected, always connected, or is connected as a function of transmit and receive mode of operation in addition to or as an alternative to the imaging mode connection for the tuning circuit 16. Any of various combinations of tuning circuits 14, 16 and associated modes may be provided, such as using one or more tuning circuits as a function of one type of imaging mode, using one or more different or same tuning circuits as a function of transmit versus receive mode operation and/or having other tuning circuits responsive to yet other modes of operation.

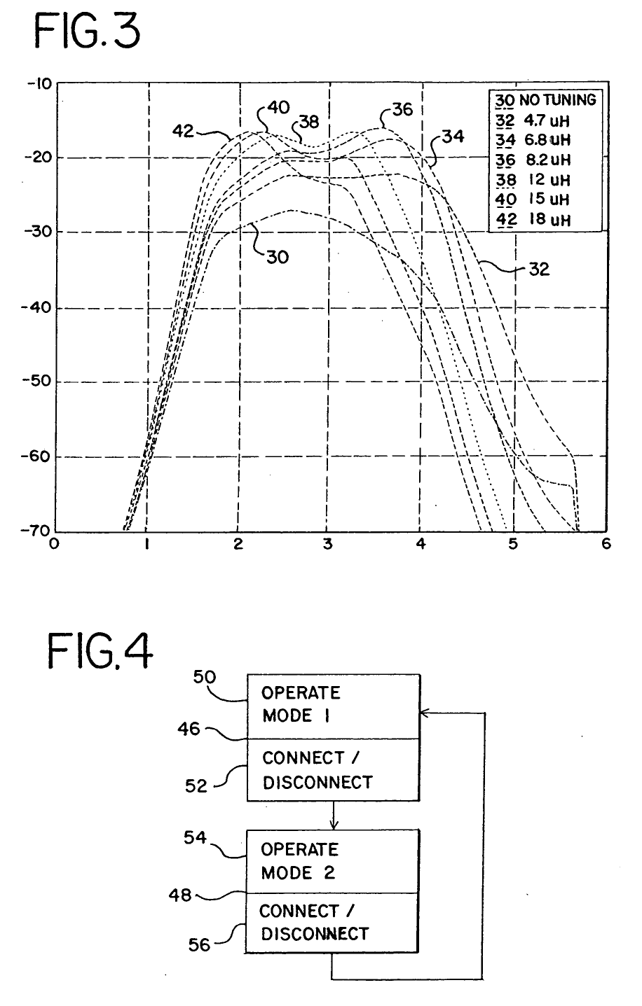

By selecting the tuning circuits 14, 16, the frequency response or spectral content of the transducer element 12 is altered as a function of mode. FIG. 3 shows one example simulation of the spectral content of a 3 Mhz transducer tailored using the circuit shown in FIG. 2. The inductances shown in FIG. 2 are tuned between the inductor L1 in one mode of operation and the inductance given by L1L2/L2+L1 in another mode of operation. The equation above may be a simplification, such as where magnetic coupling is provided between the two inductances. L2 is selected to be less than L1. The dot dash line 30 represents the frequency or spectral content of the transducer element 12 without the tuning provided by either of the tuning circuits 14, 16. Different amounts of tuning are then shown in FIG. 3. 4.7 μH is shown at line 32, 6.8 μH is shown at line 34, 8.2 μH is shown at line 36, 12 μH is shown at line 38, 15 μH is shown at line 40 and 18 μH s shown at line 42. As the inductance increases, the center frequency of the response of the transducer 12 due to tuning is decreased. In one embodiment of FIG. 2, the inductor L1 is selected at 18 μH and the inductor L2 is selected as 10 μH. The single switch 18 is closed, connecting the tuning circuit 16, to provide 6.4 μH inductance for high frequency operation. The switch 18 is opened to provide 18 μH inductance for low frequency operation. Other inductance values, inductors, or tuning circuits may be provided. For example, a series combination of inductances may be used. One or more inductances are switchably connected in series with the transducer element 12. Lower value inductances may be used for series connection than for parallel connection. As another example, one or more inductors are connectable in either series or parallel with the transducer element. The series connection may extend high frequency operation while the shunt connection may extend low frequency operation. As yet another example, a path that bypasses the other tuning networks is switchably connected to the transducer element 12.

FIG. 4 shows one embodiment of a flow chart representing a method for mode dependent tuning of a transducer. In act 48, a single layer or multi-layer transducer element is tuned with one tuning circuit during a first mode of operation. The tuning circuit is connected between a cable and the transducer element. In act 48, the transducer element is tuned with a second tuning circuit during a second mode of operation different than the first mode of operation of act 46. The second tuning circuit is different than the first tuning circuit, such as having at least one different component or all different components. The second tuning circuit and first tuning circuit may have one or more components in common. The second tuning circuit is also connected between the cable and the transducer element, such as within a probe housing, but may be connected in the connector on the other end of the cable from the scanhead. Additional modes of operation and associated selections of tuning circuits may be provided in alternative embodiments.

In the tuning of act 46, the transducer is operated in a first mode, such as a transmit/receive mode or an imaging mode. In act 52, a circuit in the transducer probe is connected with the transducer element for the first mode of operation. For example, a resistor, a capacitor, an inductor or combinations thereof is connected with the transducer element. Other circuits may be disconnected or additionally connected during the first mode of operation.

For the tuning of act 48, the transducer element is operated in a second mode of operation, different than the first mode. For example, a receive as opposed to transmit or transmit as opposed to receive mode of operation is provided. Alternatively or additionally, a different imaging mode is provided in act 54 than for act 50. In act 56, the circuit connected in act 52 is disconnected during the second mode of operation. Disconnection is provided by opening a switch. Alternatively, the circuit is disconnected by connecting additional components to form a different circuit. In one embodiment, a completely different circuit or a circuit with some shared components is connected in act 56 and disconnected in act 52.

Acts 50 and 54 correspond to transmitting in one mode with the transducer element and then transmitting with the transducer element in a second different mode. Alternatively or additionally, transmitting is provided as one mode using a single layer element. A receiving operation is the second mode using a single layer element. Transmit and receive modes with multiple layer elements may also be used. Receiving in two different imaging modes may also be used.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

1. A transducer system for mode dependent tuning, the system comprising:
a transducer having a first element;
first and second tuning circuits electrically connectable with the first element, each of the first and second tuning circuits having at least one of: a capacitor, a resistor, an inductor and combinations thereof, the first and second tuning circuits within a transducer assembly; and
a switch operable to connect and disconnect at least one of the first and second tuning circuits to the first element.

2. The transducer system of claim 1 wherein the first element comprises a single layer element.

3. The transducer system of claim 2 further comprising:
a controller connectable with the switch, the controller operable to control the switch such that a different one of: the first tuning circuit, the second tuning circuit and both the first and second tuning circuit connects with the first element during receive operation than during transmit operation.

4. The transducer system of claim 1 wherein the first tuning circuit comprises a first inductor and the second tuning circuit comprises a second inductor in parallel with the first inductor, the switch being in series with the first and second inductors.

5. The transducer system of claim 1 wherein the switch comprises a diode.

6. The transducer system of claim 1 wherein the switch comprises a transistor.

7. The transducer system of claim 1 further comprising:
a controller connectable with the switch, the controller operable to control the switch such that the first tuning circuit connects with the first element for transmit operation in a first mode and such that the second tuning circuit connects with the first element for transmit operation in a second mode different than the first mode.

8. The transducer system of claim 1 further comprising:
a controller connectable with the switch, the controller operable to control the switch such that the first tuning circuit connects with the first element for transmit operation in a first mode and such that the first tuning circuit is disconnected with the first element for transmit operation in a second mode different than the first mode, the second tuning circuit connected with the first element in both the first and second modes.

9. The transducer system of claim 1 further comprising:
a controller connectable with the switch, the controller operable to control the switch such that a same one of: the first tuning circuit, the second tuning circuit and both the first and second tuning circuit connects with the first element during both transmit and receive operation of a first mode and a different one of: the first tuning circuit, the second tuning circuit and both the first and second tuning circuit during both transmit and receive operation of a second mode different than the first mode.

10. The transducer system of claim 9 wherein the first mode comprises a B-mode and the second mode comprises a flow mode.

11. The transducer system of claim 1 further comprising:
a cable connected with the switch, the first and second tuning circuits and the switch connectable between the cable and the first element.

12. A transducer system for mode dependent tuning, the system comprising:
a transducer element;
first and second tuning circuits within a transducer probe and electrically connectable with the transducer element;
a switch operable to connect and disconnect at least one of the first and second tuning circuits to the transducer element; and
a controller operable to control the switch to connect the at least one of the first and second tuning circuits to the transducer element during both transmit and receive operations of a first mode and to disconnect the at least one of the first and second tuning circuits from the transducer element during both transmit and receive operations of a second mode different than the first mode.

13. The transducer system of claim 12 wherein the first mode is a B-mode and the second mode is a flow mode.

14. The transducer system of claim 12 wherein the first and second modes are different ones of: B-mode, flow mode, harmonic mode, M-mode, spectral Doppler mode, three-dimensional and combinations thereof.

15. The transducer system of claim 12 wherein the controller is operable to control the switch during real-time imaging sequentially scanning in both the first and second modes.

16. The transducer system of claim 12 wherein the switch is operable to disconnect the second tuning circuit from the transducer element when the first tuning circuit is connected to the transducer element and to disconnect the first tuning circuit from the transducer element with the second tuning circuit is connected to the transducer element.

17. A transducer system for mode dependent tuning, the system comprising:
a single layer transducer element within a transducer assembly;
first and second tuning circuits electrically connectable with the single layer transducer element; and
a switch within the transducer assembly and operable to connect and disconnect at least one of the first and second tuning circuits to the single layer transducer element.

18. The transducer system of claim 17 further comprising:
a controller operable to control the switch to connect the at least one of the first and second tuning circuits to the single layer transducer element during one of transmit and receive operations and to disconnect the at least one of the first and second tuning circuits from the single layer transducer element during another one of the transmit and receive operations.

19. A transducer system for mode dependent tuning, the system comprising:
a transducer element;
first and second tuning circuits electrically connectable with the transducer element;
a switch operable to connect and disconnect at least one of the first and second tuning circuits to the transducer element; and
a controller operable to control the switch to connect the at least one of the first and second tuning circuits to the transducer element during transmit operation of a first mode and to disconnect the at least one of the first and second tuning circuits from the transducer element during transmit operation of a second mode different than the first mode.

20. A method for mode dependent tuning of a transducer, the method comprising:
(a) operating in a first mode;
(b) connecting a first circuit in a transducer probe to a transducer element during (a);
(c) operating in a second mode; and
(d) disconnecting the first circuit from the transducer element during (c);
wherein (a) and (c) comprise one of:
(i) transmitting in the first mode and transmitting in the second mode;
(ii) transmitting in the first mode, the transducer element being a single layer element, and receiving in the second mode.

21. The method of claim 20 wherein (a) and (c) comprise (i).

22. The method of claim 20 wherein (a) and (c) comprise (ii).

23. The method of claim 20 further comprising:
(e) connecting a second circuit to the transducer element during (c); and
(f) disconnecting the second circuit from the transducer element during (a).

24. The method of claim 23 wherein (b) comprises connecting the first circuit having at least one of: a first resistor, a first capacitor, a first inductor and combinations thereof;
wherein (e) comprises connecting the second circuit having at least one of: a second resistor, a second capacitor, a second inductor and combinations thereof.

25. A method for mode dependent tuning of a transducer, the method comprising:
(a) tuning a single layer transducer element with a first tuning circuit during a first mode, the first tuning circuit connected between a cable and the signal layer transducer element; and
(b) tuning the single layer transducer element with a second tuning circuit during a second mode different than the first mode, the second tuning circuit different than the first tuning circuit, and the second tuning circuit connected between the cable and the single layer transducer element.

26. The method of claim 25 further comprising:
(c) switching between (a) and (b) with diodes.

27. The transducer system of claim 12 wherein the second mode is a Doppler mode.

28. The transducer system of claim 1 wherein the first tuning circuit comprises a first inductor and the second tuning circuit comprises a second inductor in series with the first inductor.

29. A transducer system for mode dependent tuning, the system comprising:
a transducer element;
a first active component electrically connectable with the transducer element;
a switch operable to connect and disconnect the first active component to the transducer element; and
a controller operable to control the switch to perform at least one of:
connect the first active component to the transducer element during transmit operation of a first mode and to disconnect the first active component from the transducer element during transmit operation of a second mode different than the first mode; and
connect the first active component to the transducer element during receive operation of the first mode and to disconnect the first active component from the transducer element during receive operation of the second mode different than the first mode.

30. The system of claim 29 wherein the controller is operable to connect the first active component to the transducer element during both transmit and receive operations of the first mode and to disconnect the at least one of the first and second tuning circuits from the transducer element during both transmit and receive operations of the second mode different than the first mode, wherein the first and second modes are different ones of: B-mode, flow mode, harmonic mode, M-mode, spectral Doppler mode, three-dimensional and combinations thereof.

31. The system of claim 29 wherein the active component comprises at least one of a transistor, an amplifier and a processor.
